(19) United States
(12) Patent Application Publication
(10) Pub. No.: US 2026/0014306 A1
(43) Pub. Date: Jan. 15, 2026

(54) FILTRATION APPARATUS WITH SOLID GASKET AND STATIONARY MEMBRANE

(71) Applicant: Nephrodite, Inc., Atlanta, GA (US)

(72) Inventors: Farbod Sedaghati, Alpharetta, GA (US); Hiep Thieu Nguyen, Chandler, AZ (US); Arnav Jain, Suwanee, GA (US)

(21) Appl. No.: 19/268,098

(22) Filed: Jul. 14, 2025

Related U.S. Application Data
(60) Provisional application No. 63/670,259, filed on Jul. 12, 2024.

(51) Int. Cl. A61M 1/36

(52) U.S. Cl. CPC A61M 1/362263

(57) ABSTRACT

This invention discloses a novel filtration apparatus with a stationary filtration membrane.

### CROSS-REFERENCE TO RELATED PATENT APPLICATIONS

This application claims priority to and the benefit of U.S. Provisional Patent Application Ser. No. 63/670,259 filed Jul. 12, 2024, which is incorporated herein in its entirety by reference.

### FIELD OF THE INVENTION

The present invention relates generally to healthcare, and more particularly to medical devices that filter fluids through a semi-permeable membrane.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Membranes are typically developed for stationary applications where they experience one directional pressure or load. In the past, filtration devices have utilized membranes located within a moveable gasket that creates pressure differentials through its movement to cause fluid to move through the membrane, which thereby filters out, e.g., impurities from the fluid. However, the membrane's movement in such devices creates significant dynamic stresses on the membrane, which can lead to its rupture. As a result, the membrane area is restricted in an effort to reduce such stresses and the resulting risks of membrane failure.

Therefore, a heretofore unaddressed need exists in the art to address the aforementioned deficiencies and inadequacies.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows a prior art filtration device.

FIG. 2 shows a prior art filtration device.

FIG. 3 shows an embodiment of the invention.

### SUMMARY OF THE INVENTION

This invention in certain aspects discloses an apparatus that reduces dynamic stresses upon a semi-permeable membrane through its location on a fixed gasket in a filtration vessel. One such vessel is the Holly implantable dialysis device described in common-owned U.S. patent application Ser. No. 17/101,265.

In one embodiment, a stationary gasket comprises a semi-permeable membrane located between two chambers. Fluid from the first chamber is filtered through the membrane into the second chamber through the flexion and relaxation of a non-filtering solid gasket located on one side of the device. The advantages of this stationary filtration membrane apparatus include the substantial reduction of dynamic stresses experienced by the ultrafiltration membrane. In addition, the stationary ultrafiltration membrane apparatus also operates through a lower applied pressure required for range of motion due to only one movable gasket, a shorter cycle period (time to complete one full inlet and outlet cycle) which reduces clotting risk, and an increase in filtration area of at least 2x compared to prior art designs incorporating the ultrafiltration membrane into a moveable gasket.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

Prior art implantable medical device systems, including the device disclosed in the common-owned U.S. Pat. No. 10,744,250 (the “'250 Patent) and U.S. Pat. No. 10,874,786, (the “'786 Patent), utilize at least one ultrafiltration membrane within the devices to perform dialysis. Such membrane is contained within one or more gaskets that flex when pumps operate to draw fluid, e.g., blood on one side and dialysate on the other, into their respective chambers and empty such chambers within the device.

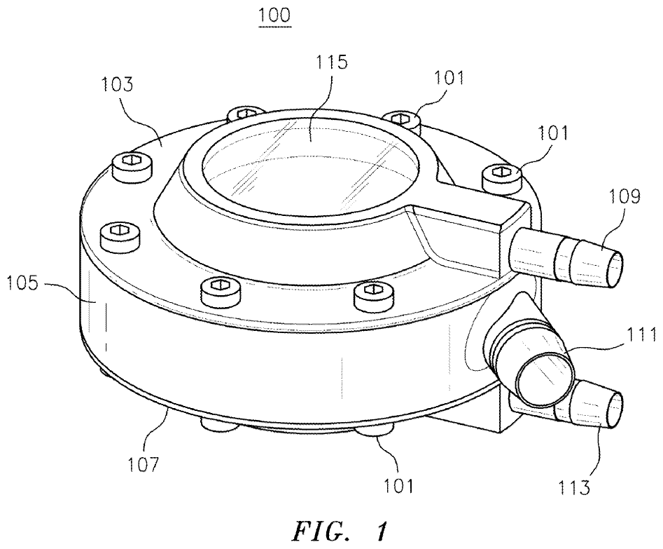

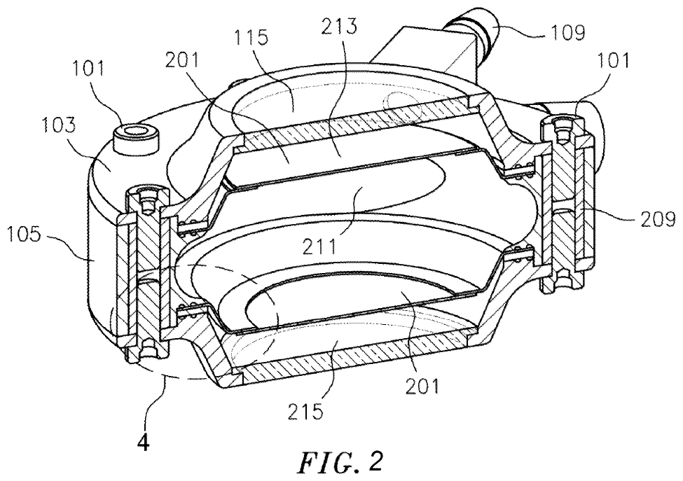

FIG. 1 shows this prior art device system. Referring now to the structure of the prior art filtration unit, as shown in the perspective view of FIG. 1, the filtration unit 100 includes a middle housing 105 sealingly connected to both a top housing 103 and a bottom housing 107 by a plurality of fasteners 101. FIG. 2 depicts a cross-sectional view of the filtration unit 100, the top housing 103 and the bottom housing 107 contain the top dialysis chamber 213 and the bottom dialysis chamber 215, respectively, and the middle housing 105 contains the blood chamber 211. The top housing 103 and the bottom housing 107 have openings that provide a means to sealingly affix flexible outer membranes 115 to the top housing 103 and the bottom housing 107. The flexible outer membranes 115 each contain an ultrafiltration membrane 201. As the pump causes the top dialysis chamber 213 and bottom dialysis chamber 215 to be filled with dialysate from a reservoir, the flexible outer membranes 115 expand causing ultrafiltration membranes 201 to be moved toward the blood chamber 211, increasing the transit of waste products from the blood chamber 211 and into the top dialysate chamber 213 and bottom dialysate chamber 215, as well as forcing the cleansed blood from the blood chamber 211.

As flexible outer membranes 115 move towards the blood chamber, they exert significant force on the fluid which transforms into kinetic energy. So, during the inlet cycle, the two flexible outer membranes 115 experience high stresses applied to the membrane supports and stress concentration points. Also, during the outlet cycle where the blood chamber is filled with pressurized blood caused by the patient's circulatory system, the flow that has a dynamic force tends to push the two membranes away. This force, which is called the inertia effects, is exerted on the membrane 115 surface, and is transferred to the regions in the flexible outer membrane 115 that support the ultrafiltration membrane 201. During cyclic motion of the outer membranes 115, fibers near the stress concentrations zones of the ultrafiltration membrane 201 experience high stress levels which eventually leads to membrane failure.

Prior art devices, such as shown in FIG. 1 and FIG. 2, sought to reduce the risk of such failure by limiting the surface area of the membrane. An aspect of the invention described herein allows the use of a much larger surface area in the ultrafiltration membrane 301 as a non-moving gasket.

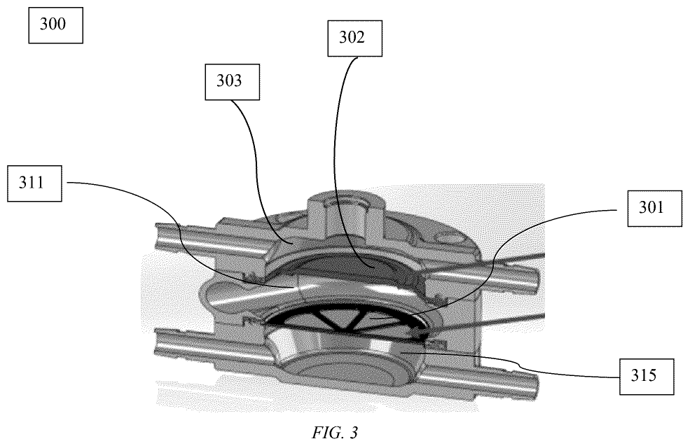

FIG. 3 shows an embodiment of the invention. This embodiment comprises a device 300 with a larger ultrafiltration membrane 301 operating as a non-movable gasket between the blood chamber 311 and bottom dialysate chamber 315 and a separate movable membrane 302 that, through its relaxation and flexion, causes blood to move into and out of blood chamber 311. In particular, a hydraulic fluid, which may also serve as a dialysate, is pumped from the upper hydraulic chamber 303, causing movable membrane 302 to relax and blood to enter blood chamber 311. As blood enters the blood chamber 311, it interacts with bottom dialysate chamber 315 across ultrafiltration membrane 301, causing impurities to move from the blood into the dialysate. Thereafter, hydraulic fluid is pumped back into upper hydraulic chamber 303, causing movable membrane 302 to flex towards blood chamber 311, causing blood to move from blood chamber 311. After a predetermined number of cycles, the contaminated dialysate in bottom dialysate chamber 315 is removed and replaced with fresh dialysate. In another embodiment, the contaminated dialysate is stored in a separate reservoir and utilized as a hydraulic fluid to fill upper hydraulic chamber 303.

The foregoing descriptions of an exemplary embodiment of the invention have been presented only for the purposes of illustration and description and are not intended to be exhaustive or to limit the invention to the precise forms disclosed. Many modifications and variations are possible in light of the above teaching.

The embodiment was chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is not limited by the foregoing description and the exemplary embodiments described therein.

What is claimed is:

1. A dialysis device implantable in a patient for dialysis, comprising a filtration unit, the filtration unit comprising:
at least one dialysis chamber for containing and/or circulating dialysate;
at least one blood chamber for containing and/or circulating blood of the patient, disposed on at least one dialysis chamber and being in communication with the at least one dialysis chamber;
at least one hydraulic chamber for containing a hydraulic fluid and further comprising a moveable membrane; and
at least one ultrafiltration membrane disposed between the at least one dialysis chamber and the at least one blood chamber such that the blood contained in the blood chamber is in contact with one surface of the at least one ultrafiltration membrane that faces the blood chamber, the dialysate fluid contained in the at least one dialysis chamber is in contact with another surface of the at least one ultrafiltration membrane that faces the at least one dialysis chamber, and the blood in the at least one blood chamber and the dialysate in the at least one dialysis chamber operably interact with each other for dialysis,
wherein the at least one hydraulic chamber operates to fill and empty the at least one blood chamber; and
wherein the at least one ultrafiltration membrane operates as a non-moveable gasket between the at least one blood chamber and the at least one dialysis chamber.

2. The dialysis device of claim 1, wherein the hydraulic fluid is dialysate.

3. The dialysis device of claim 2, wherein the dialysate has been contaminated through its use in the dialysis chamber.
